(19) United States
(12) Patent Application Publication
(10) Pub. No.: US 2024/0277329 A1
(43) Pub. Date: Aug. 22, 2024

(54) SELF-CINCHING ADJUSTABLE LOOP SUTURE BUTTON AND METHODS

(71) Applicant: Christian N. Anderson, Nashville, TN (US)

(72) Inventor: Christian N. Anderson, Nashville, TN (US)

(21) Appl. No.: 18/582,250

(22) Filed: Feb. 20, 2024

Related U.S. Application Data
(60) Provisional application No. 63/446,767, filed on Feb. 17, 2023.

(51) Int. Cl. A61B 17/04

(52) U.S. Cl. CPC A61B 17/0401; A61B 2017/0404; A61B 2017/0414

(57) ABSTRACT

A suture button and a self-cinching adjustable loop suture construct that approximates tissue to a desired location during ligament repair or reconstruction is provided. The suture button includes first and second bridges and corresponding passages positioned to attach a suture construct to the button using a hitch around the first and second bridges. A recess is defined in the bottom surface of the button to accommodate a portion of the suture construct spanning between the two bridges without interfering with the underlying bone, tissue or structure against which the suture button is positioned. The suture button is configured to provide an anchor such that tension may be applied to the self-cinching adjustable loop suture construct to tighten the adjustable loops of the construct and apply tension to a ligament.

### CROSS-REFERENCES TO RELATED APPLICATIONS

This application claims benefit of and priority to U.S. Provisional Application No. 63/446,767 filed Feb. 17, 2023 entitled Self-Cinching Adjustable Loop Suture Button and Methods, all of which is hereby incorporated by reference in its entirety.

### TECHNICAL FIELD

The present disclosure relates to devices and methods for orthopedic surgery, and more particularly to suture button anchors to secure suture devices and associated methods to repair and reconstruct ligaments and tissue.

### BACKGROUND

Conventional suture button anchor devices and associated methods for fixation of flexible suture material are difficult or ineffective for use with certain types of procedures and suture constructs. Some orthopedic surgical procedures for fixation of tissue or internal bracing utilize suture constructs including one or more self-cinching sections wherein suture material passes through itself either axially or transversely to maintain tension on the suture construct during or after the procedure. Such self-cinching suture constructs may be used in a variety of surgical procedures, and self-cinching suture constructs may be used in combination with knots or independently without the use of knots to tighten the suture construct.

Self-cinching suture constructs in some applications are not compatible with conventional suture button anchor devices. What is needed then, are improvements in suture buttons, suture constructs, suture assemblies and associated methods, all for repairing and reconstructing tissue in orthopedic surgical procedures.

### BRIEF SUMMARY

The present disclosure provides improved suture button devices, self-cinching adjustable loop suture constructs, suture assemblies and associated methods for performing orthopedic surgery.

A suture button configured for use with self-cinching adjustable loop suture constructs includes a top surface, a bottom surface, a first end and a second end. A center passage is defined through the suture button between the first and second ends in some embodiments. A first passage is defined through the suture button between the center passage and the first end, and a second passage is defined through the suture button between the center passage and the second end. A first bridge extends between the center passage and the first passage; and a second bridge extends between the center passage and the second passage. A self-cinching adjustable loop suture construct is attached to the first and second bridges in a hitch. The suture button provides an anchor for tensioning the suture construct.

The suture button devices, self-cinching adjustable loop constructs, suture assemblies and associated methods disclosed herein may be used in numerous applications such as but not limited to repair or reconstruction of the anterior cruciate ligament (ACL), other ligament repair or reconstruction, syndesmotic repair, acromioclavicular (AC) joint repair or reconstruction, medial patellofemoral ligament (MPFL) repair or reconstruction or other surgical procedures.

An objective of the present disclosure in some embodiments is to provide an improved suture button for use with self-cinching adjustable loop suture constructs for orthopedic surgery.

A further objective of the present disclosure in some embodiments is to provide a self-cinching adjustable loop suture construct for ligament fixation during orthopedic surgery.

Another objective of the present disclosure in some embodiments is to provide a suture assembly including a suture button and a self-cinching adjustable loop suture construct disposed on the suture button for orthopedic surgery.

A further objective of the present disclosure in some embodiments is to provide a method of tightening a suture construct.

Yet another objective of the present disclosure in some embodiments is to provide a method of forming a self-cinching section of a suture construct.

Another objective of the present disclosure in some embodiments is to provide a method of assembling a suture assembly including a suture button and a self-cinching adjustable loop suture construct.

Numerous other objects, advantages and features of the present disclosure will be readily apparent to those of skill in the art upon a review of the drawings and description as set forth below.

### DESCRIPTION OF THE DRAWINGS

FIG. 1 illustrates a top perspective view of an embodiment of a suture button.

FIG. 2 illustrates a side view of the embodiment of a suture button of FIG. 1.

FIG. 3 illustrates an end view of the embodiment of a suture button of FIG. 1.

FIG. 4 illustrates a bottom perspective view of an embodiment of a suture button of FIG. 1.

FIG. 5 illustrates a side cross-sectional perspective view of the embodiment of a suture button of FIG. 1.

FIG. 6. illustrates a perspective cross-sectional view of the embodiment of a suture button of FIG. 1.

FIG. 7 illustrates a bottom plan view of the embodiment of a suture button of FIG. 1.

FIG. 8 illustrates a top plan view of the embodiment of a suture button of FIG. 1.

FIG. 9 illustrates a top perspective view an embodiment of a suture button configured for syndesmosis.

FIG. 10 illustrates a side view of the embodiment of a suture button of FIG. 9.

FIG. 11 illustrates an end view of the embodiment of a suture button of FIG. 9.

FIG. 12 illustrates a bottom perspective view of the embodiment of a suture button of FIG. 9.

FIG. 13 illustrates a side cross-sectional view of the embodiment of a suture button of FIG. 9.

FIG. 14 illustrates a perspective cross-sectional view of the embodiment of a suture button of FIG. 9.

FIG. 15 illustrates a bottom perspective view of the embodiment of a suture button of FIG. 9.

FIG. 16 illustrates a partial cross-sectional view of an embodiment of a suture assembly including a suture button and a self-cinching adjustable loop suture construct with parallel adjustable loops.

FIG. 17 illustrates a partial cross-sectional view of an embodiment of a suture assembly including a suture button and a self-cinching adjustable loop suture construct with parallel adjustable loops and first and second locking loops.

FIG. 18 illustrates a partial cross-sectional view of an embodiment of a suture assembly including a suture button and a self-cinching adjustable loop suture construct.

FIG. 19 illustrates a partial cross-sectional view of an embodiment of a suture assembly including a suture button and a self-cinching adjustable loop suture construct with independent adjustable loops.

FIG. 20 illustrates a partial cross-sectional view of an embodiment of a suture assembly including a suture button and a self-cinching adjustable loop suture construct with interlocking adjustable loops.

FIG. 21 illustrates an embodiment of a suture assembly including a suture button and a self-cinching adjustable loop suture construct including a free continuous loop disposed on the adjustable loops.

FIG. 22 illustrates an embodiment of a suture assembly including a suture button and a self-cinching adjustable loop suture construct including a free continuous loop disposed on the adjustable loops and a shuttling suture disposed on the continuous loop.

FIG. 23 illustrates an embodiment of a suture assembly including a suture button and a self-cinching adjustable loop suture construct including an anchor and a shuttling suture disposed on the adjustable loops.

FIG. 24 illustrates an embodiment of a suture assembly including a suture button and a self-cinching adjustable loop suture construct including a free continuous loop disposed on the adjustable loops, an anchor disposed on the continuous loop, and a shuttling suture disposed on the anchor.

FIG. 25 illustrates an embodiment of a configuration for a self-cinching sleeve or self-cinching section on the suture construct.

FIG. 26 illustrates an embodiment of a configuration for a self-cinching sleeve or self-cinching section on the suture construct.

FIG. 27 illustrates an embodiment of a configuration for a self-cinching sleeve or self-cinching section on the suture construct.

FIG. 28 illustrates an embodiment of a configuration for a self-cinching sleeve or self-cinching section on the suture construct.

FIG. 29 illustrates an embodiment of a configuration for a self-cinching sleeve or self-cinching section on the suture construct.

FIG. 30 illustrates an embodiment of a configuration for a self-cinching sleeve or self-cinching section on the suture construct.

FIG. 31 illustrates an embodiment of a configuration for a self-cinching sleeve or self-cinching section on the suture construct.

FIG. 32 illustrates an embodiment of a suture assembly including a suture button and a self-cinching adjustable loop suture construct positioned for ACL reconstruction.

FIG. 33 illustrates an embodiment of a suture assembly including a suture button and a self-cinching adjustable loop suture construct positioned for ACL reconstruction.

FIG. 34 illustrates a detail view of an embodiment of a suture assembly including a suture button and a self-cinching adjustable loop suture construct positioned for ACL reconstruction.

FIG. 35 illustrates an embodiment of a suture assembly including a suture button and a self-cinching adjustable loop suture construct including flat suture tape looped around a reconstruction graft.

FIG. 36 illustrates an embodiment of a suture assembly including a suture button and a self-cinching adjustable loop suture construct configured for ankle syndesmosis repair.

FIG. 37 illustrates an embodiment of a suture assembly including a suture button and a self-cinching adjustable loop suture construct configured for ankle syndesmosis repair.

FIG. 38 illustrates an embodiment of a suture assembly including a suture button and a self-cinching adjustable loop suture construct with first and second threading sutures configured for ACL repair.

FIG. 39 illustrates an embodiment of a suture assembly including a suture button and a self-cinching adjustable loop suture construct configured for ACL repair.

FIG. 40 illustrates an embodiment of a suture assembly including a suture button and a self-cinching adjustable loop suture construct configured for ACL repair.

### DETAILED DESCRIPTION

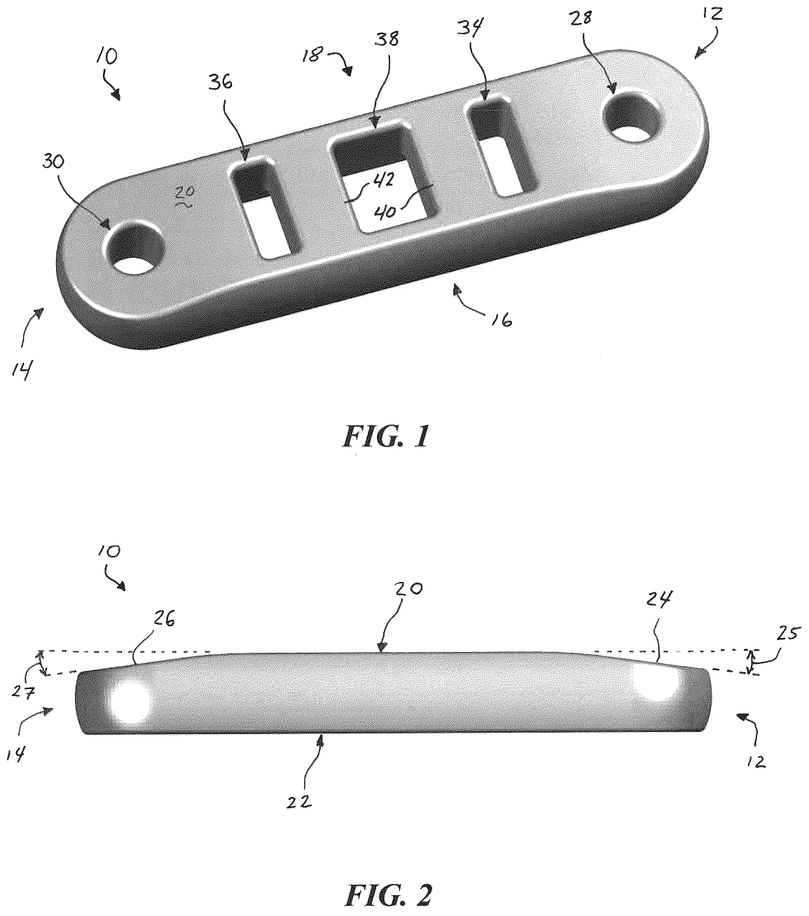

The present disclosure provides various embodiments of improved self-cinching adjustable loop button devices and associated methods for use during orthopedic surgery. Referring to FIGS. 1-8, an embodiment of a suture button 10 configured for use with self-cinching adjustable loop suture constructs is illustrated. Although suture button 10 is described herein as being configured for use with a self-cinching adjustable loop suture construct, suture button 10 may be used with other types of suture constructs, including knotted suture constructs, in various applications.

Referring to FIG. 1, suture button 10 has an elongated profile and includes a first end 12 and a second end 14 opposite first end 12 positioned at the longitudinal ends of suture button 10. First side 16 is located between first and second ends 12, 14, and a second side 18 is located opposite first side 16 between first and second ends 12, 14. Button 10 also includes a top surface 20 and a bottom surface 22, shown in FIG. 2.

First and second ends 12, 14 each include a rounded profile in two dimensions, forming a convex rounded shape in some embodiments. First end 12 includes a rounded shape extending from first side 16 to second side 18, forming a semi-circular shape in some embodiments. First end 12 also includes a rounded profile from top surface 20 to bottom surface 22, as shown in FIG. 2. Second end 14 includes a rounded shape extending from first side 16 to second side 18, forming a semi-circular shape in some embodiments. Second end 14 also includes a rounded profile from top surface 20 to bottom surface 22, as shown in FIG. 2. The rounded profiles of first and second ends 12, 14 aid in the passage of suture button 10 through tissue or through a tunnel drilled or reamed through bone. Additionally, suture button 10 includes an elongated profile with a width between first and second sides 16, 18 less than length between first and second ends 12, 14. In some embodiments, the aspect ratio of length to width is between about two and about ten. In further embodiments, the aspect ratio of length to width is between about three and about six. In additional embodiments, the aspect ratio of length to width is between about three and about four.

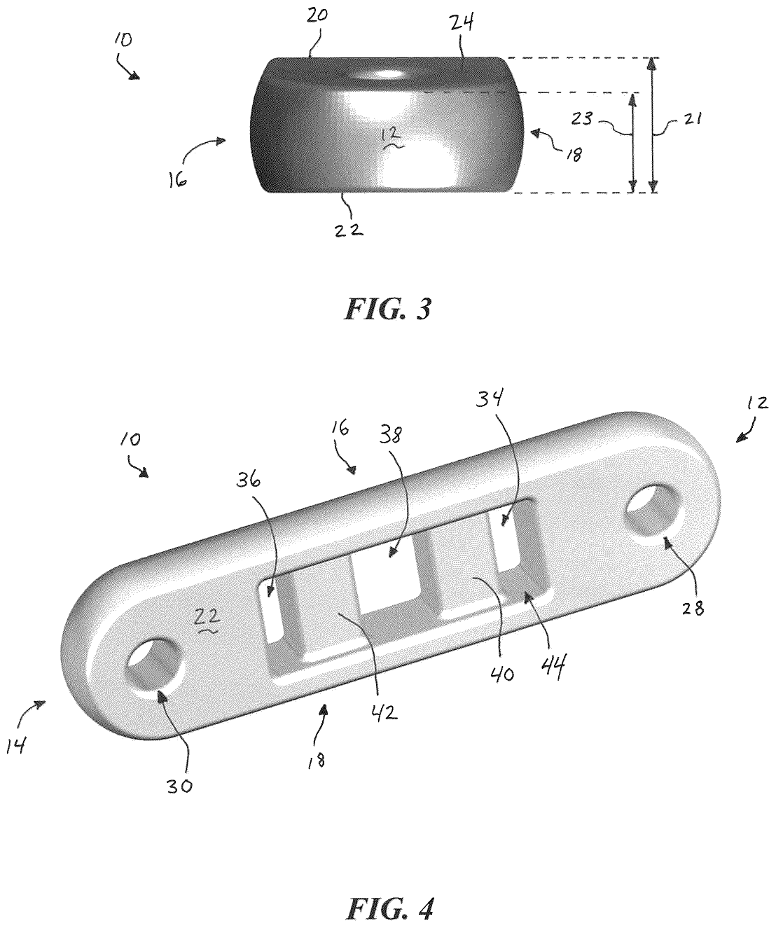

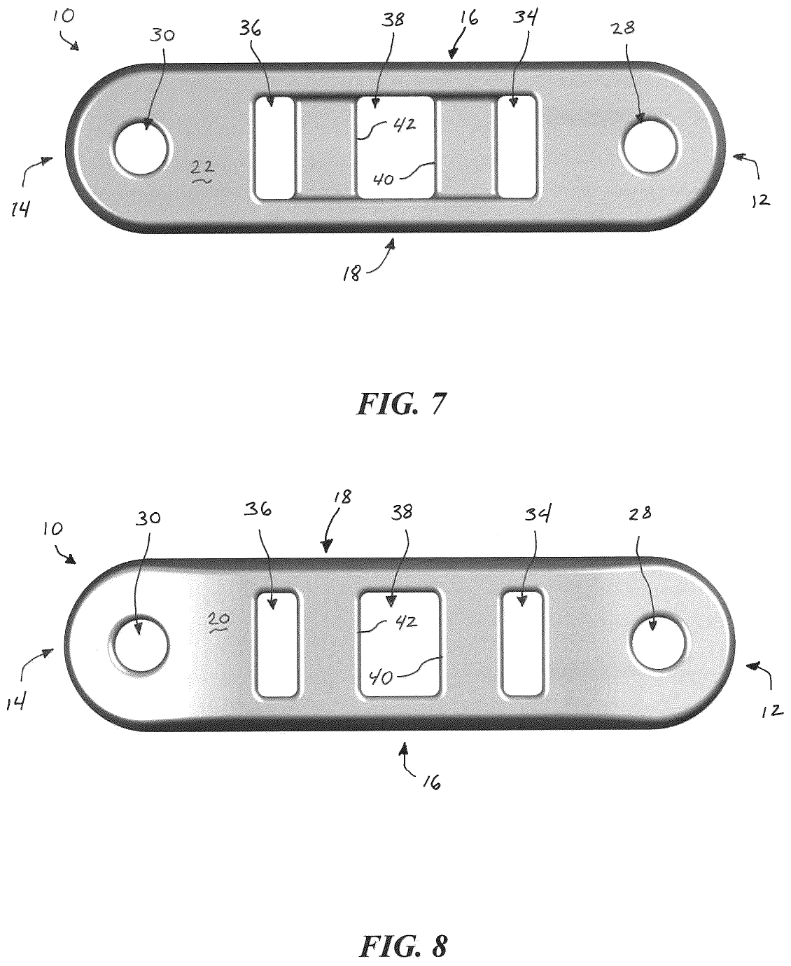

Referring to FIG. 2 and FIG. 3, suture button 10 also includes a first ramp 24 on first end 12 and a second ramp 26 on second end 14. The middle portion of suture button 10 on top surface 20 includes a substantially flat surface in some embodiments. As the top surface 20 approaches first end 12, first ramp 24 includes an inclined surface sloping downwardly toward bottom surface 22, forming a tapered first end. First ramp 24 includes a substantially flat, angled surface having a first ramp angle 25. First ramp angle 25 is between about one degree and about thirty degrees in some embodiments. First ramp 24 provides a tapered profile that further enhances passage of suture button 10 through tissue or through a tunnel in bone. Additionally, as the top surface 20 approaches second end 14, second ramp 26 includes an inclined surface sloping downwardly toward bottom surface 22, forming a tapered second end. Second ramp 26 includes a substantially flat, angled surface having a second ramp angle 27. Second ramp angle 27 is between about one degree and about thirty degrees in some embodiments. Second ramp 26 provides a tapered profile that further enhances passage of suture button 10 through bone or tissue and is less likely to rub against or damage soft tissue. As shown in FIG. 7 and FIG. 8, in some embodiments, suture button 10 is symmetric about a lateral axis passing through both the longitudinal and lateral axes of suture button 10 which makes suture button reversible in that it may be pulled through tissue or a bone tunnel from first end 12 or second end 14.

Referring further to FIG. 2 and FIG. 3, in some embodiments, bottom surface 22 is substantially flat, forming a planar surface. Suture button 10 includes a body thickness 21, and first end 12 includes an end thickness 23 less than body thickness 21 due to the downward sloping profile of first ramp 24. Second end 14 includes a similar end thickness less than body thickness 23. In some embodiments, the end thickness 23 of first end 12 and second end 14 are substantially equal such that the first and second ramps 24, 26 include identical but opposing shapes and slope profiles.

Referring again to FIG. 1, suture button 10 includes a plurality of clearance holes or passages defined through suture button 10 from top side 20 to bottom side 22. Each clearance hole or passage provides an opening for passing one or more suture strands or constructs for attachment of suture button 10 to tissue or for passing suture button 10 through tissue or a bone tunnel. A first hole 28 is defined through suture button 10 near first end 12. First hole 28 is defined through first ramp 24 in some embodiments. First hole 28 includes a cylindrical hole having rounded opening edges at top surface 20 and bottom surface 22. Each rounded hole or passage opening described herein is provided to prevent chafing or wear of a suture strand passing through the hole or passage. A second hole 30 is defined through suture button 10 near second end 14. Second hole 30 is defined through second ramp 26 in some embodiments. Second hole 30 includes a cylindrical hole having rounded opening edges at top surface 20 and bottom surface 22.

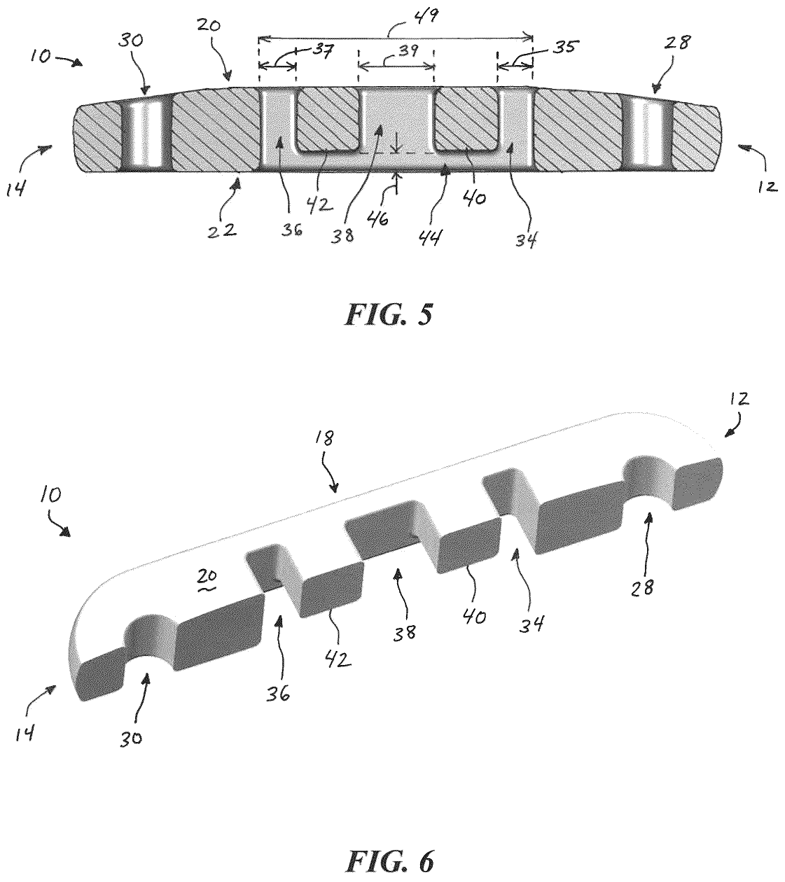

A center passage 38 is defined through suture button 10 along the lateral centerline of suture button 10 in some embodiments. Center passage 38 includes a rectangular profile with rounded interior corners, as shown in FIG. 8 in some embodiments. The rectangular profile of center passage 38 makes center passage 38 adapted to accommodate either rounded suture strands, such as solid or hollow core braided suture material, or flat suture strands such as suture tape, or alternatively to accommodate flat-to-round or round-to-flat suture strand material. Center passage 38 includes a center passage length 39, shown in FIG. 5. Center passage length 39 is less than center passage width in the lateral dimension in some embodiments.

A first passage 34 is defined through suture button 10 between center passage 38 and first hole 28. First passage 34 includes a rectangular profile with rounded interior corners in some embodiments. First passage 34 has a first passage length 35 less than center passage length 39 in some embodiments. First passage 34 has a first passage width in the lateral direction greater than first passage length 35 in some embodiments.

A second passage 36 is defined through suture button 10 between center passage 38 and second hole 30. Second passage 36 includes a rectangular profile with rounded interior corners in some embodiments. Second passage 36 has a second passage length 37 less than center passage length 39 in some embodiments. Second passage 36 has a second passage width in the lateral direction greater than second passage length 37 in some embodiments.

A first bridge 40 is disposed from first side 16 to second side 18 between first passage 34 and center passage 38. First bridge 40 includes a substantially rectangular cross-sectional profile including rounded corners, as shown in FIG. 5 and FIG. 6. First bridge 40 provides structure for securing a suture strand to suture button 10 by passing a suture strand around first bridge 40 through center passage 38 and first passage 34.

A second bridge 42 is disposed from first side 16 to second side 18 between second passage 36 and center passage 38. Second bridge 42 includes a substantially rectangular cross-sectional profile including rounded corners, as shown in FIG. 5 and FIG. 6. Second bridge 42 provides structure for securing a suture strand to suture button 10 by passing a suture strand around second bridge 42 through center passage 38 and second passage 36.

Referring to FIG. 4, FIG. 5, FIG. 6 and FIG. 7, in some embodiments, suture button 10 includes a recess 44 defined on bottom surface 22. Recess 44 is defined as a rectangular cutout defined in the bottom surface 22 with rounded interior corners in some embodiments. Recess 44 includes a recess depth 46 defined as the spacing between bottom surface 22 and the lower surfaces of first and second bridges 40, 42. In some embodiments, recess 44 is co-extensive with the end walls of first and second recesses 34, 36 defined as recess length 49, as shown in FIG. 5. In some embodiments, recess 44 may be defined by providing first and second bridges 40, 42 each having a thickness less than the body thickness 21. Recess 44, or alternatively, first and second bridges 40, 42 having a thickness less than body thickness 21, provides a clearance space on the bottom surface 22 of suture button 10 to accommodate the thickness of one or more suture strands passing around first and/or second bridges 40, 42. As shown in FIG. 5 and FIG. 6, in some embodiments, the walls of recess 44 extending through suture button 10 include substantially planar, rectangular walls with rounded edges to reduce chafing or wear on suture strand material passing through first, second or center passage 34, 36, 36.

The embodiment of suture button 10 shown in FIGS. 1-8 includes a suture button adapted in some applications for repair or reconstruction of the anterior cruciate ligament (ACL) during orthopedic surgery. Suture button 10 is generally configured such that bottom surface 22 may be positioned against the femoral cortex adjacent a bone tunnel, and a suture construct extends from bottom surface 22 through the bone tunnel toward the ligament. The top surface 22 is configured to face away from the femoral cortex when suture button 10 is implanted. As such, suture button 10 provides an anchor for fixation of a suture construct for ligament repair or reconstruction.

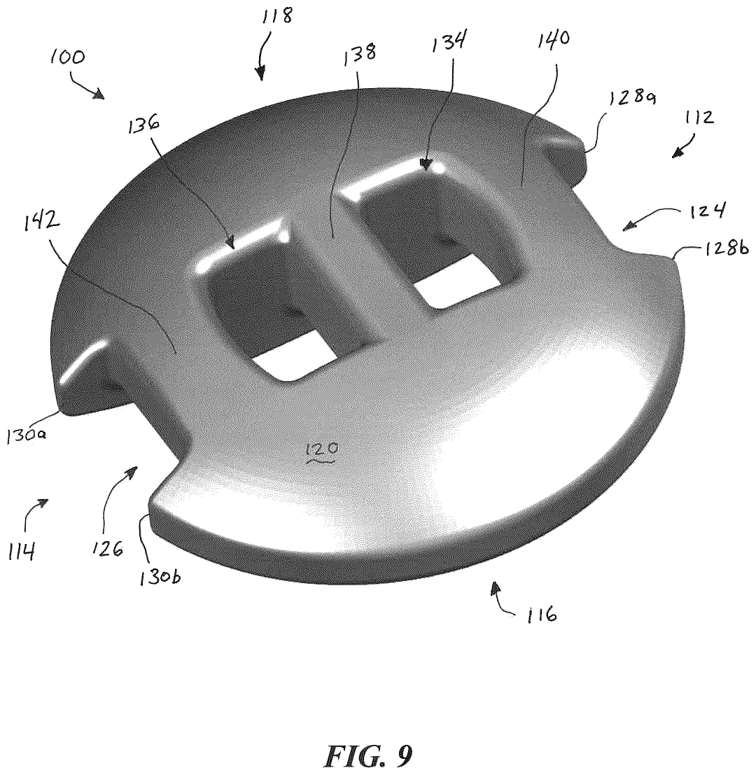

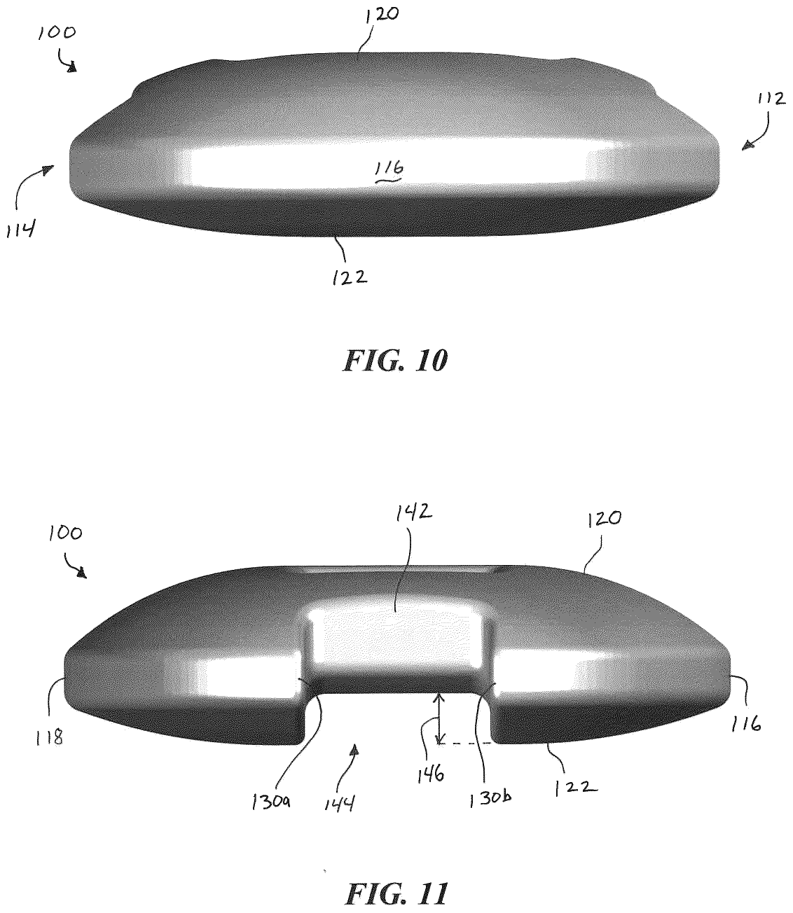

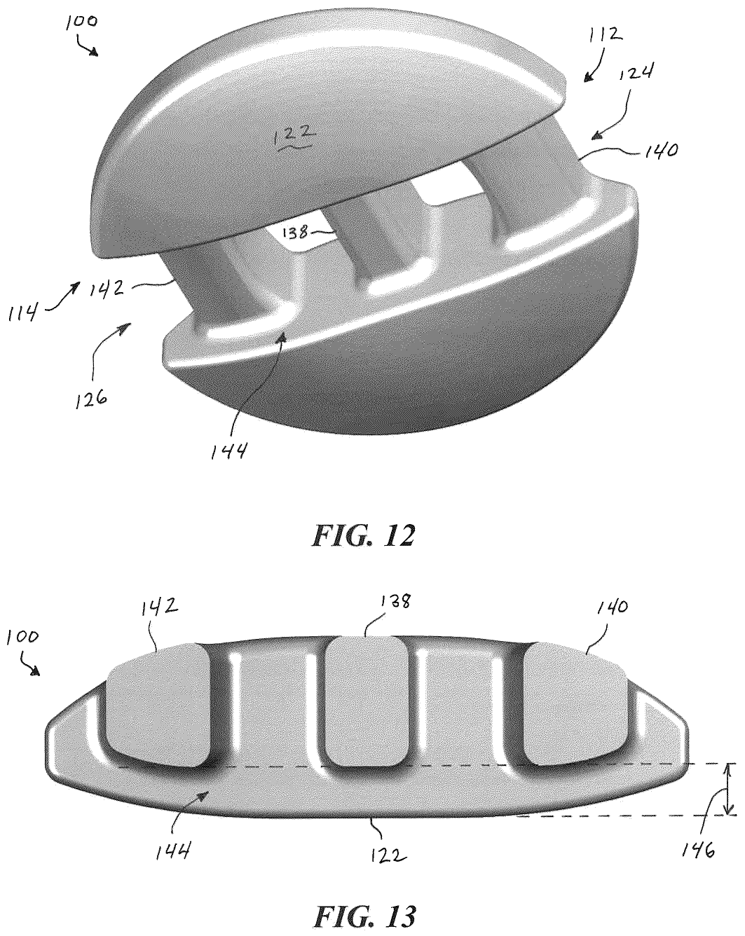

Referring to FIGS. 9-15, an alternative embodiment of a suture button 100 configured for use in syndesmotic fixation is provided. Suture button 100 includes a round profile having a convex curved top surface 120 and also a convex curved bottom surface 122. Bottom surface 122 is curved to seat in a corresponding concave socket on a metal fixation plate used for syndesmosis, as opposed to the substantially flat bottom surface 22 of suture button 10. Additionally, suture button 100 includes a substantially round outer profile between first edge 116, second edge 118, first end 112 and second end 114. In some embodiments, button 100 includes a circular outer profile. In other embodiments, button 100 includes an elliptical or elongated circular profile.

A first passage 134 is defined through suture button 100 from top side 120 to bottom side 122 near first end 112. First passage 134 defines a clearance passage through which one or more suture strands may be passed. A second passage 136 is defined through suture button 100 from top side 120 to bottom side 122 near second end 114. Second passage 134 defines a clearance passage through which one or more suture strands may be passed. A center bridge 138 is disposed on suture button 100 between first and second passages 134, 136.

Referring further to FIG. 9, suture button 100 includes a first recess 124 defined in first end 112 and a second recess 126 defined in second end 114. First and second recess 124, 126 each provide a clearance socket for passage of one or more suture strands such that the suture strand material does not extend past the outer profile of suture button 10 when positioned in first or second recess 124, 126.

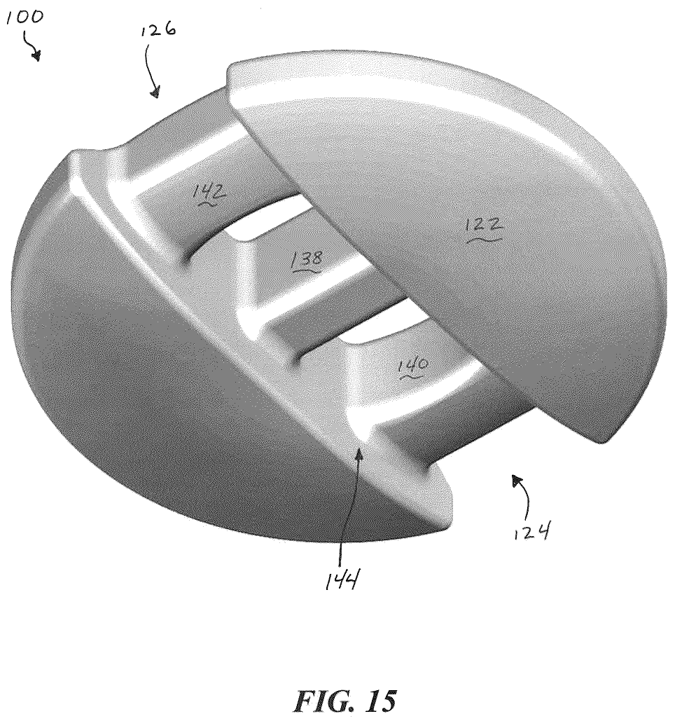

Referring to FIGS. 11-13, a bottom recess 144 is defined in bottom surface 122 in some embodiments. Bottom recess 144 is defined from first end 112 to second end 114. Bottom recess 144 includes a bottom recess depth 146 defined as the recess depth between bottom surface 122 and the lower edges of center bridge 138, first bridge 140 and second bridge 142. Bottom recess 144 includes a longitudinal channel defined in bottom surface 122 from first recess 124 to second recess 126, as shown in FIG. 15. Bottom surface 144 includes substantially parallel, planar interior walls with rounded outer edges in some embodiments.

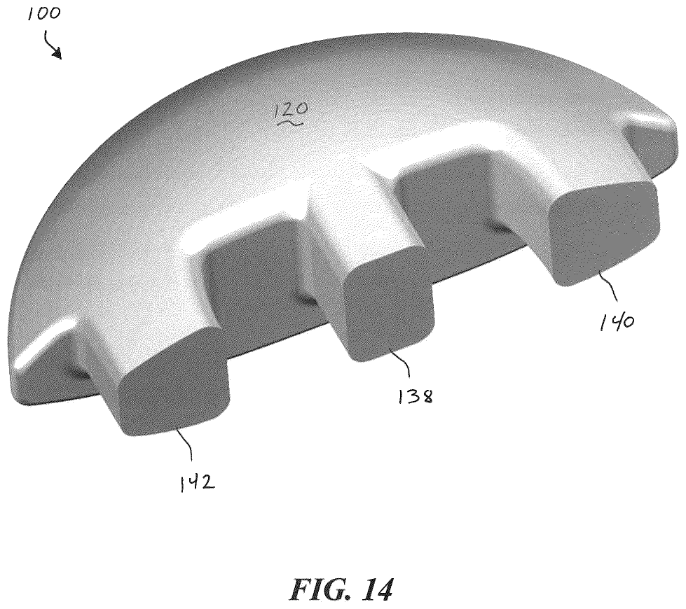

Referring to FIG. 13 and FIG. 14, in some embodiments, first bridge 140 and second bridge 142 each include a substantially trapezoidal cross-sectional profile, and center bridge 138 includes a substantially rectangular cross-sectional profile. Each bridge includes rounded corners to reduce wear or chafing of a suture strand passing through a passage or wrapped around a bridge.

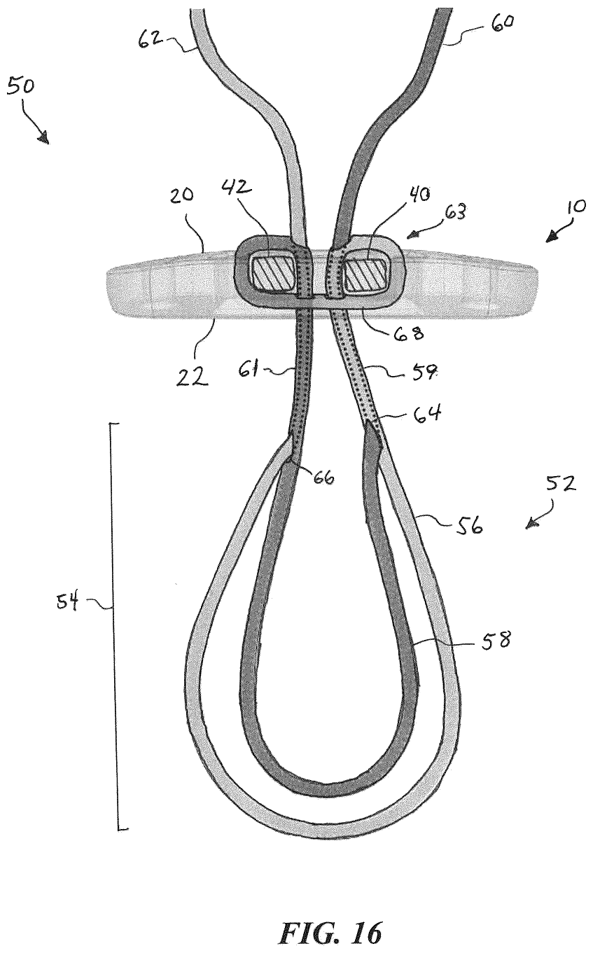

Referring to FIG. 16 and FIG. 18, an embodiment of a suture assembly 50 including a suture button 10 and a self-cinching adjustable loop suture construct 52 is shown. Suture assembly 50 is adapted for ACL repair or reconstruction in some embodiments. Suture construct 52 includes a single strand of suture material, including but not limited to hollow-core suture, braided suture, flat suture, or suture tape. Suture construct 52 includes an adjustable main loop 54 comprising a first strand 56 and a second strand 58. First and second strands 56, 58 are portions of a single suture forming a double loop. Main loop 54 is adjustable in that the length of main loop 54 may be shortened by pulling on first and second tag ends 60, 62.

Main loop 54 includes a first self-cinching section, or self-cinching sleeve 59 and a second self-cinching section, or self-cinching sleeve 61 positioned substantially parallel to the first self-cinching sleeve 59. The terms self-cinching section and self-cinching sleeve may be used interchangeably depending on the specific configuration of the self-cinching configuration employed in the suture construct. First self-cinching sleeve 59 is formed on first strand 56, and second strand 58 enters a first sleeve opening 64 on first strand 56. Second strand 58 passes through the interior hollow core of first strand 56 toward suture button 10. Second strand 58 exits the sleeve at or near suture button 10 forming a first tag end 60 projecting away from top surface 20. During use, first tag end 60 may be pulled, causing second strand 58 to slide inside the hollow interior core of first strand 56 inside first self-cinching sleeve 59. When tension is applied, the first self-cinching sleeve 59 clamps down on second strand 58, thereby locking first and second strands in a fixed axial position relative to each other inside first self-cinching sleeve 59. As more tension is applied, the clamping force increases. Such a configuration allows the suture button 10 to be pulled tight against bone or tissue, such as the femoral cortex during ACL repair or reconstruction.

Similarly, second self-cinching sleeve 61 is formed on second strand 58, and first strand 56 enters a second sleeve opening 66 on second strand 58. First strand 56 passes through the interior hollow core of second strand 58 toward suture button 10. First strand 56 exits the sleeve at or near suture button 10, forming second tag end 62. During use, second tag end 62 may be pulled, causing first strand 56 to slide inside the hollow interior core of second strand 58 inside second self-cinching sleeve 61. When tension is applied, the second self-cinching sleeve 61 clamps down on first strand 56, thereby locking first and second strands in a fixed axial position relative to each other inside the second self-cinching sleeve 61. As more tension is applied the clamping force increases. Such a configuration allows the suture button 10 to be pulled tight against bone or tissue, such as the femoral cortex during ACL repair or reconstruction.

In some embodiments, first and second tag ends 60, 62 are both pulled simultaneously to tighten main loop 54 to a desired length and tension. Main loop 54 is formed when the suture material is attached to suture button 10. The suture material forms a hitch 63 around first bridge 40 and second bridge 42. A cross strand 68 spans the underside of first and second bridges 40, 42. Cross strand 68 is positioned inside recess 44 near bottom surface 22 of suture button 10. Recess 44 is dimensioned such that cross strand 68 is positioned entirely within recess 44 such that cross strand 68 does not protrude below bottom surface 22 in some embodiments. The first self-cinching sleeve 59 along with the interior second strand 58 both pass alongside cross strand 68 in some embodiments. In other embodiments, first self-cinching sleeve 59 along with the interior second strand 58 puncture and pass transversely through the suture material of cross strand 68 through first transverse passage 70 in cross strand 68. Similarly, the second self-cinching sleeve 61 along with the interior first strand 56 both pass alongside cross strand 68 in some embodiments. In other embodiments, second self-cinching sleeve 61 along with interior first strand 56 puncture and pass transversely through the suture material of cross strand 68 through second transverse passage 72 in cross strand 68.

Referring further to FIG. 18, in some embodiments, the self-cinching adjustable loop suture construct includes multiple locking modes. First, hitch 63 formed around first and second bridges 40, 42 secures the suture construct to suture button 10. Second, the self-cinching sleeves 59, 61 provide locking of the suture construct in the axial direction when tightened by pulling first and second tag ends 60, 62. Third, passage of the suture material through first and second transverse passages 70, 72 in cross strand 68 provides radial tightening of the suture construct, circumferentially compressing the suture material further limiting suture slippage through the axial self-cinching sleeves and improving pullout strength of the adjustable loops. Additionally, main loop 54 includes a first adjustable loop formed by first strand 56 and a second adjustable loop formed by second strand 58. The first and second adjustable loops forming main loop 54 provide redundant tensioning to the tissue to be repaired or reconstructed.

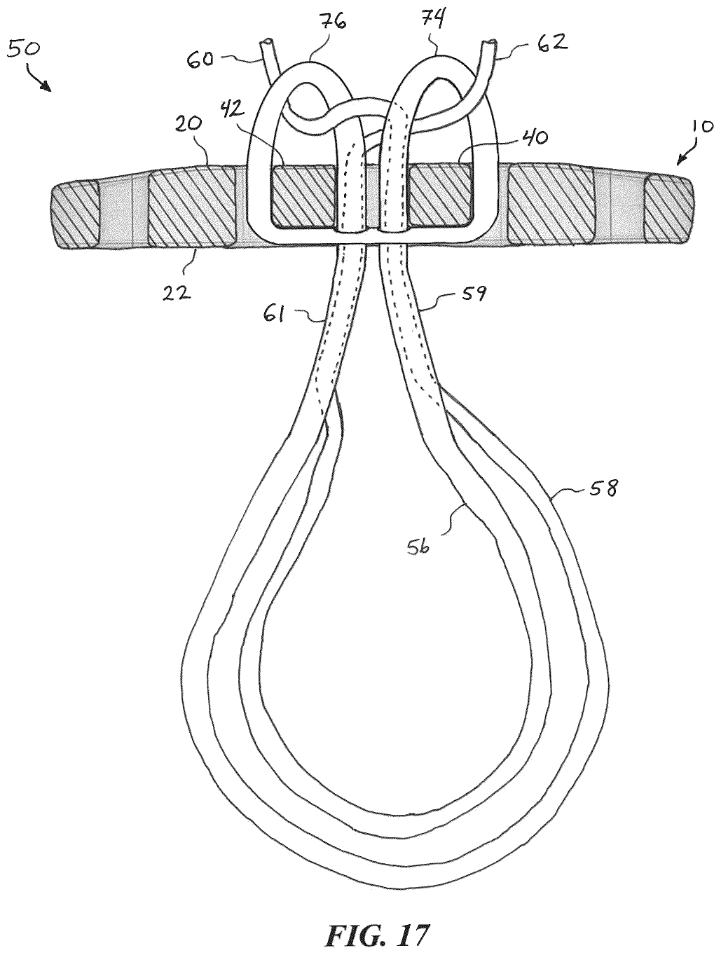

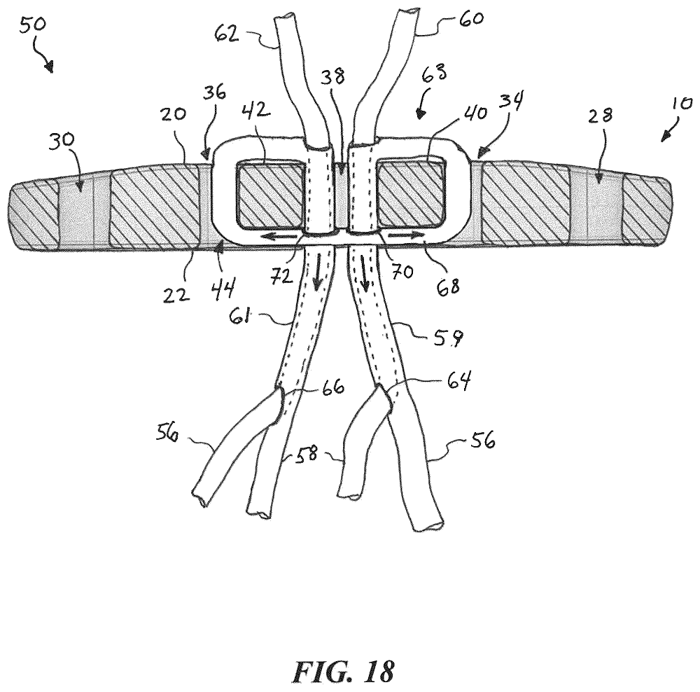

Referring to FIG. 17, in some embodiments, suture assembly 50 includes a suture construct 52 and a suture button 10 configured as described with respect to FIG. 16 and FIG. 17 and including an additional locking mode. In such embodiments, first tag end 60 exits first self-cinching sleeve 59 and passes under a portion of second strand 58 forming a first locking loop 76 on the upper side of second bridge 42 on top surface 20 of suture button 10. First tag end 60 is clamped against the upper surface of second bridge 42 by first locking loop 76 when the suture construct is tightened, thereby further locking the suture construct in tension. Similarly, second tag end 62 exits second self-cinching sleeve 61 and passes under a portion of first strand 56 forming a second locking loop 74 on the upper side of first bridge 40 on top surface 20 of suture button 10. Second tag end 62 is clamped against the upper surface of first bridge 40 by second locking loop 74 when the suture construct is tightened, thereby further locking the suture construct in tension.

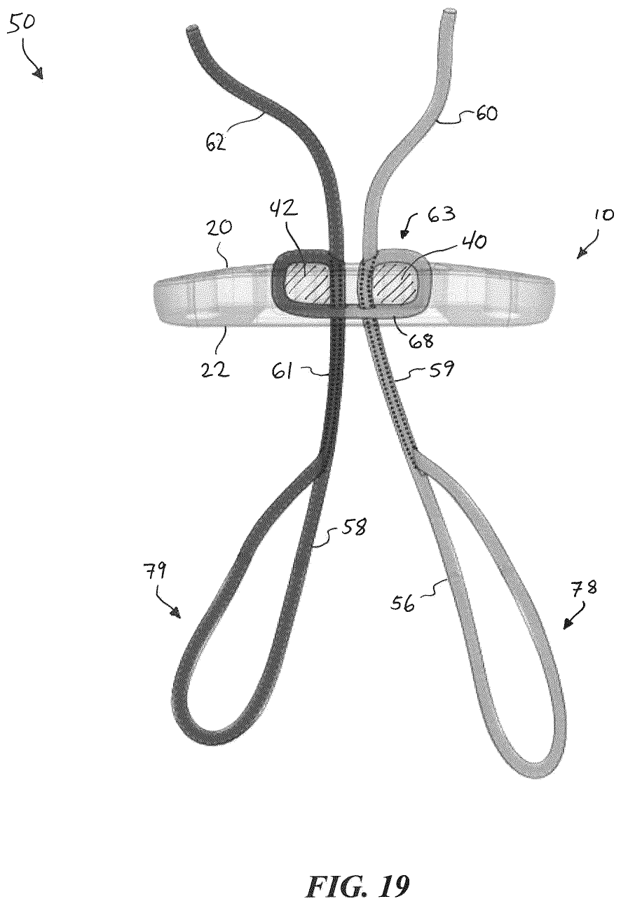

Referring to FIG. 19, an alternative embodiment of a suture assembly 50 includes independent adjustable loops. First adjustable loop 78 is formed on first strand 56 extending from bottom surface 22 of suture button 10. Second adjustable loop 79 is formed on second strand 58 extending from bottom surface 22 of suture button 10. First and second adjustable loops 78, 79 may be attached independently to tissue for repair or reconstruction, or may be independently attached to another suture construct or loop. First strand 56 forms first adjustable loop 78 and passes back through first self-cinching sleeve 59 in itself, and first tag end 60 exits from first self-cinching sleeve 59 extending away from top surface 20 on suture button 10. Second strand 58 forms second adjustable loop 79 and passes back through second self-cinching sleeve 61 in itself, and second tag end 62 exits from second self-cinching sleeve 61 extending away from top surface 20 on suture button 10.

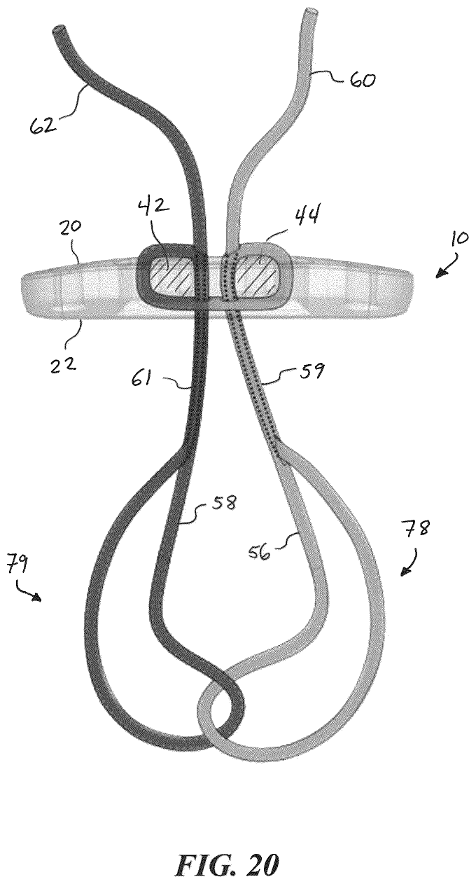

Referring to FIG. 20, an alternative embodiment of a suture assembly 50 includes interlocking adjustable loops. First adjustable loop 78 is formed on first strand 56 extending from bottom surface 22 of suture button 10. Second adjustable loop 79 is formed on second strand 58 extending from bottom surface 22 of suture button 10. First adjustable loop 78 loops through second adjustable loop 79 such that first adjustable loop 78 pulls against second adjustable loop 79 when tightened. First strand 56 forms first adjustable loop 78 and passes back through first self-cinching sleeve 59 in itself, and first tag end 60 exits from first self-cinching sleeve 59 extending away from top surface 20 on suture button 10. Second strand 58 forms second adjustable loop 79 and passes back through second self-cinching sleeve 61 in itself, and second tag end 62 exits from second self-cinching sleeve 61 extending away from top surface 20 on suture button 10.

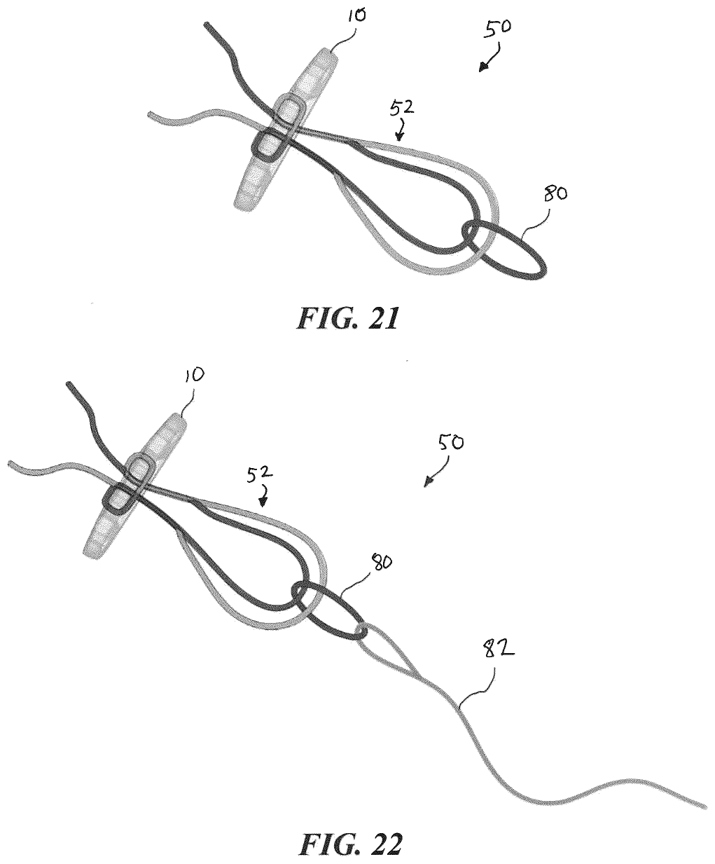

Referring to FIGS. 21-24, various embodiments of suture assemblies 50 are shown with different attachments on the adjustable loop. Each attachment includes a structure that is secured to the adjustable loop of the suture construct for attaching tissue or another suture construct or for providing an anchor for engaging bone or tissue.

Referring to FIG. 21, an embodiment of a suture assembly 50 includes a suture button 10 and a self-cinching adjustable loop suture construct 52 disposed on suture button 10. A free continuous loop 80 is positioned on the adjustable parallel loops of suture construct 52. The first and second strands of suture construct 52 may travel freely through continuous loop 80, and in some embodiments, continuous loop 80 provides a pulley against which first and second strands may be pulled to tighten the suture construct 52. In such embodiments, suture construct 52 may include any configuration described herein.

Referring to FIG. 22, an embodiment of a suture assembly 50 includes a suture button 10 and a self-cinching adjustable loop suture construct 52 disposed on suture button 10. A free continuous loop 80 is positioned on the adjustable parallel loops of suture construct 52. The first and second strands of suture construct 52 may travel freely through continuous loop 80, and in some embodiments, continuous loop 80 provides a pulley against which first and second strands may be pulled to tighten the suture construct 52. A shuttling suture 82 is disposed on continuous loop 80. Shuttling suture 82 may be used to pass a portion of the suture construct 52 through tissue or a bone tunnel. In such embodiments, suture construct 52 may include any configuration described herein.

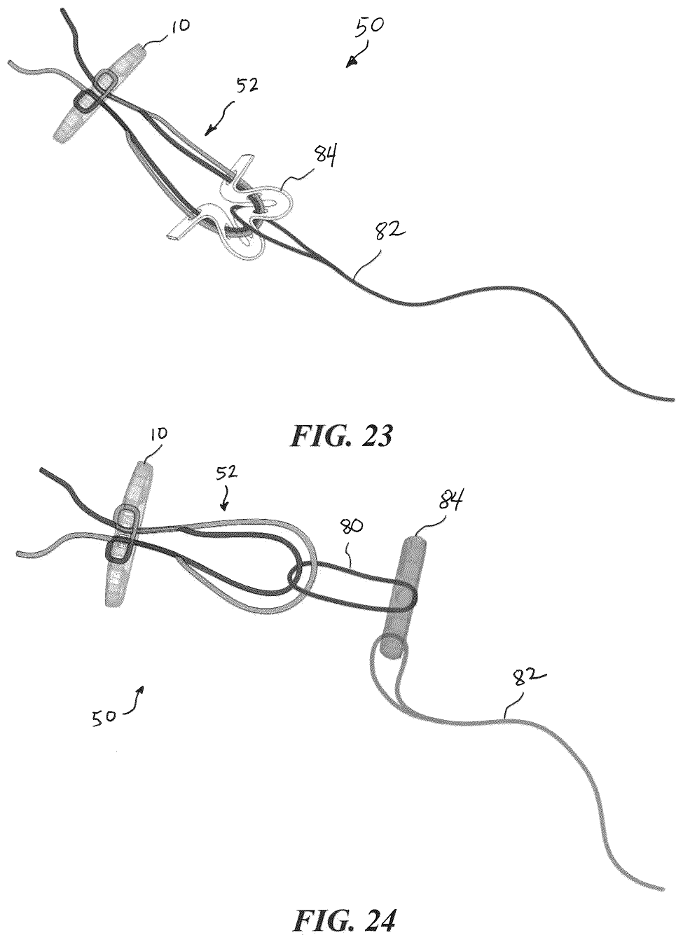

Referring to FIG. 23, an embodiment of a suture assembly 50 includes a suture button 10 and a self-cinching adjustable loop suture construct 52 disposed on suture button 10. An anchor 84 is positioned on the adjustable parallel loops of suture construct 52. The first and second strands of suture construct 52 may travel freely through anchor 84, and in some embodiments, anchor 84 provides a pulley against which first and second strands may be pulled to tighten the suture construct 52. Anchor 84 as shown in FIG. 23 includes a soft anchor in the form of flexible material such as suture tape, wherein the first and second strands of the adjustable loops are passed transversely through the anchor material. A shuttling suture 82 is disposed on the adjustable loops around anchor 84. Shuttling suture 82 may be used to pass a portion of the suture construct 52, including anchor 84, through tissue or a bone tunnel. In such embodiments, suture construct 52 may include any configuration described herein.

Referring to FIG. 24, an embodiment of a suture assembly 50 includes a suture button 10 and a self-cinching adjustable loop suture construct 52 disposed on suture button 10. A free continuous loop 80 is disposed on the adjustable loops. An anchor 84 is positioned on the continuous loop 80. The first and second strands of suture construct 52 may travel freely through continuous loop 80, and in some embodiments, anchor 84 provides a pulley against which first and second strands may be pulled to tighten the suture construct 52. Anchor 84 as shown in FIG. 24 includes a rigid anchor in the form of an elongated button, wherein the continuous loop 80 is secured to the anchor 84. A shuttling suture 82 is disposed on the anchor 84. Shuttling suture 82 may be used to pass a portion of the suture construct 52, including anchor 84, through tissue or a bone tunnel. In such embodiments, suture construct 52 may include any configuration described herein.

Numerous other attachments may be disposed on the adjustable loop portion of the suture construct 52. In various embodiments, the attachment may include a metal button, a sewable suture, a suture loop, a shuttling suture, a tape suture, an implant, graft, needle, tissue graft, plate, hollow suture, tape suture, suture anchor, metal screw or a bioresorbable implant, button, anchor or screw.

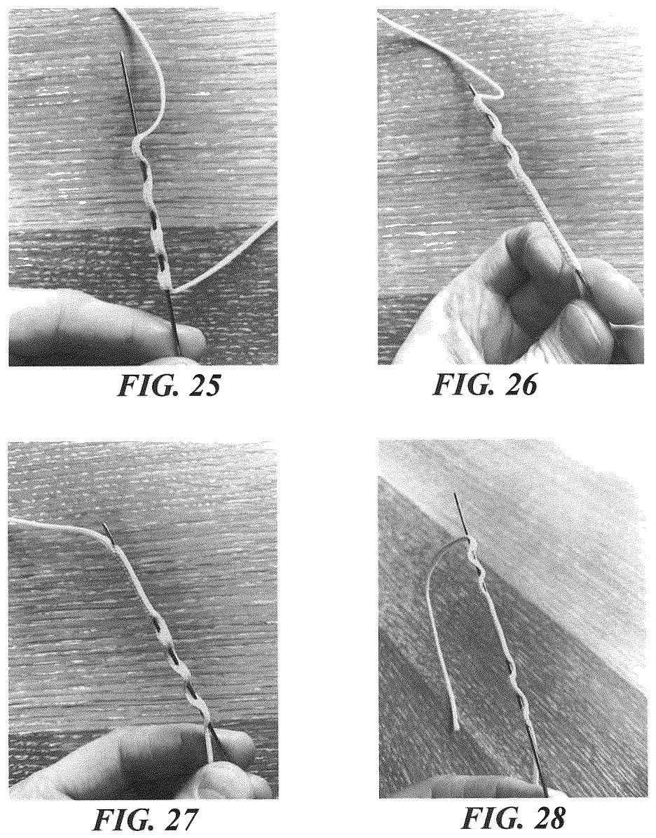

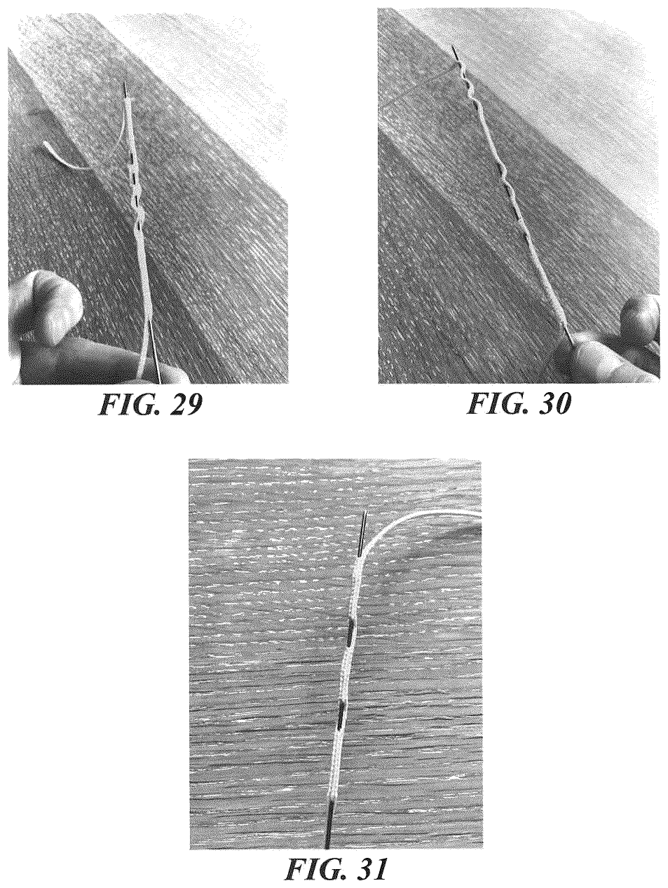

Various embodiments of self-cinching sleeve configurations are disclosed for providing the self-cinching sleeve features described herein. In a first embodiment, as shown in FIGS. 16-20, a single-pass axial self-cinching sleeve is formed by passing a suture strand axially through a hollow core suture material to form a locking mechanism. Referring to FIG. 25, in another embodiment, a suture material can be spliced in an entirely woven configuration wherein a suture strand is passed transversely through suture material using a needle. Multiple transverse passes may be provided to achieve a desired locking effect. This configuration can be used with hollow core suture material or tape suture material. Referring to FIG. 26, in another embodiment, a suture material can be spliced in a single pass-woven configuration, wherein a single pass includes passing suture material axially through the hollow core of suture material, and the woven portion includes suture material passing transversely through the body of the suture material. Referring to FIG. 27, in another embodiment, a suture material can be spliced in a woven—single pass configuration, which is a reverse of the configuration shown in FIG. 26. Referring to FIG. 28, an embodiment of a self-cinching section includes a woven—single pass—woven configuration. Referring to FIG. 29, an embodiment of a self-cinching section includes a single pass—woven—single pass configuration. Referring to FIG. 30, an embodiment of a self-cinching section includes a single pass—woven—single pass—woven configuration. Referring to FIG. 31, an embodiment of a self-cinching section includes multiple single pass sections. Numerous other combinations of single pass, woven or single pass and woven can be combined to form a self-cinching section on suture construct.

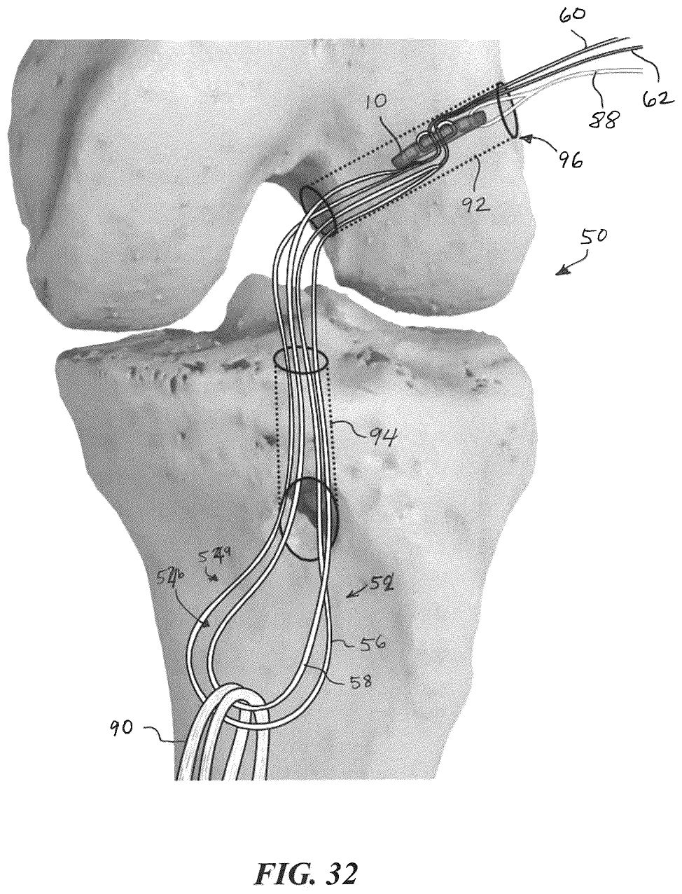

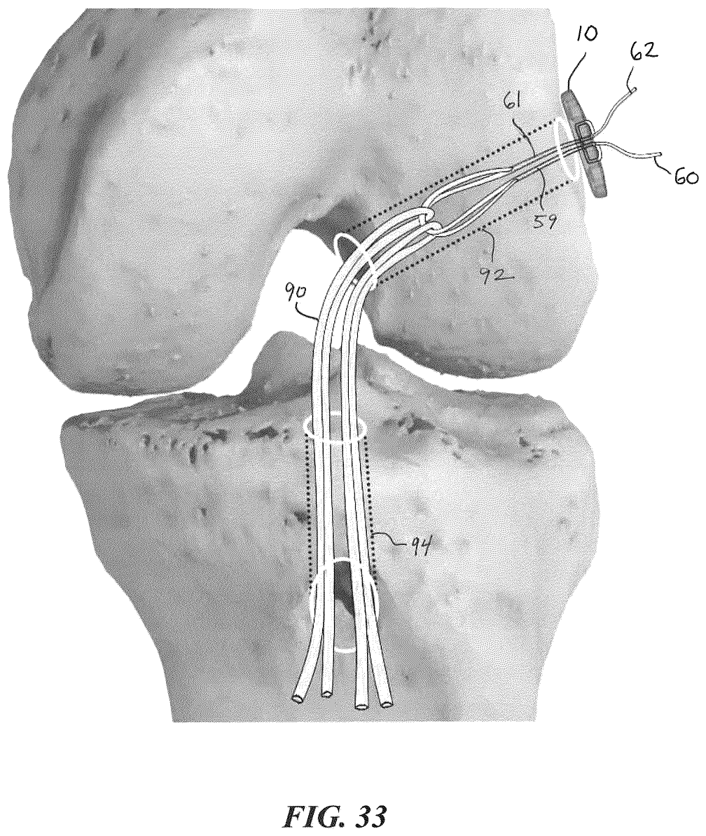

Referring to FIGS. 32-34, an embodiment of a suture assembly 50 configured for ACL reconstruction using a graft ligament is shown. Suture assembly 50 includes a suture button 10 and a self-cinching adjustable loop suture construct 52. A main loop including a first adjustable loop 54a and a second adjustable loop 54b is formed extending through a tibia bone tunnel 94. A reconstruction ligament 90 such as a hamstring graft is positioned through the first and second adjustable loops 54a, 54b. The suture button 10 with suture construct 52 attached may be pulled through a femoral tunnel 92 using a passing suture 88 disposed through first end hole 28 or second end hole 30 on suture button 10. The position of first and second end holes 28, 30 on suture button 10 allows the suture button 10 to be pulled through the femoral tunnel 92 in a longitudinal orientation. The passing suture 88 is pulled through an opening of the femoral tunnel 92 on the femoral cortex 96 until the suture button 10 exits the opening adjacent the femoral cortex 96, thereby drawing the reconstruction ligament 90 through the tibia tunnel 94, across the knee joint and toward an anatomically correct position adjacent the femur or slightly inserted into the femoral tunnel 92. Referring to FIG. 33, the suture button 10 is flipped upon exiting the opening of the femoral tunnel 92 at the femoral cortex 96 such that the bottom surface 22 lays flat against the femoral cortex 96. From this position, the distal end of the reconstruction ligament 90 may be secured at the distal opening of the tibia tunnel 94 using a suitable anchor or fixation device, and the first and second tag ends 60, 62 may be pulled to tighten the suture construct 52. When the first and second tag ends are pulled, the first and second strands 56, 58 travel through their corresponding first and second self-cinching sleeves 59, 61 and shorten the first and second adjustable loops 54a, 54b to tighten the suture construct 52 to a desired biomechanical tension and to firmly seat the suture button 10 against the femoral cortex 96. Additionally, pulling the first and second tag ends may further pull the reconstruction ligament 90 into the femoral tunnel 92 to a desired anatomic position. After tightening, the first and second tag ends may be trimmed.

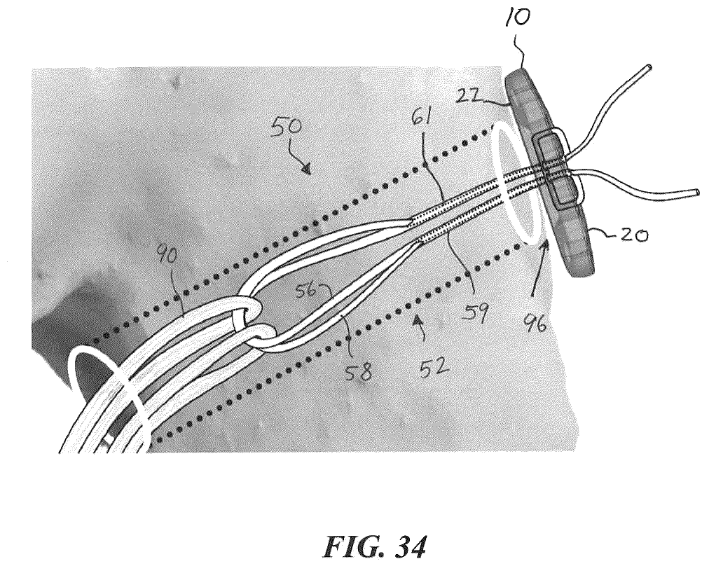

Referring to FIG. 34, in some embodiments the suture construct 52 includes a first strand 56 and a second strand 58 including flat suture tape material. As such, the flat profile of the suture tape forming first and second adjustable loops allows the suture material to lay flat against a reconstruction graft 90 to distribute the load over a wider area of the reconstruction graft 90. Such a configuration places less undesirable force on the graft than traditional round sutures and prevents the suture construct from cutting or sawing into the graft tissue or unnecessarily damaging the graft tissue.

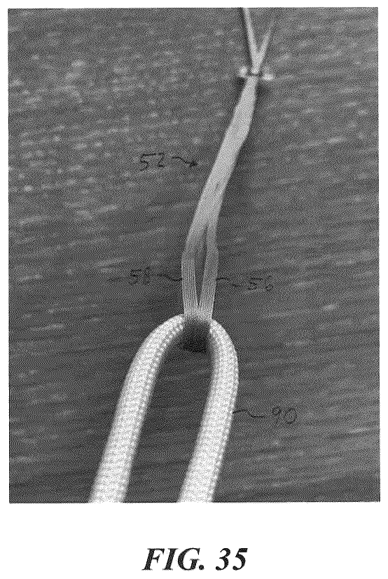

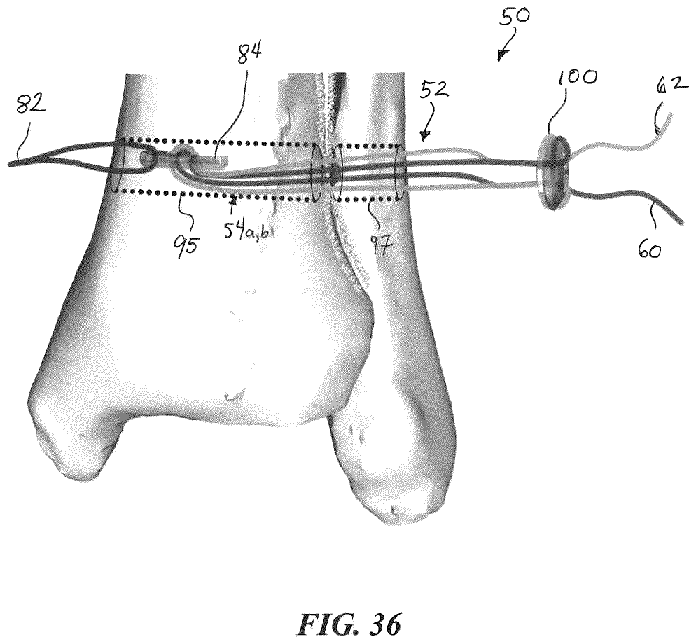

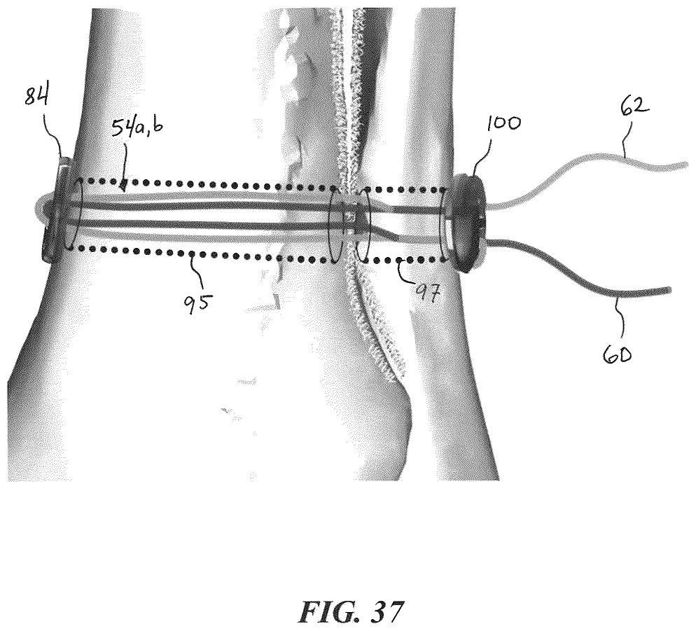

Referring to FIG. 35, in some embodiments, a suture assembly 50 configured for ankle syndesmosis repair is shown. Suture assembly 50 includes a self-cinching adjustable loop suture construct 52 and a suture button 100 similar to the embodiments shown in FIGS. 9-15. An anchor 84 is disposed on adjustable loops 54a, 54b, and a shuttling suture 82 is disposed on the anchor. Passing suture 82 is passed through a fibula tunnel 97 and a tibia tunnel 95 along with anchor 84 and suture construct 52 until anchor 84 exits an opening of tibia tunnel 95. Suture button 100 approaches an opening on the fibula tunnel or a fixation plate disposed on the tibia adjacent the opening of the tibia tunnel 95. Once anchor 84 exits the tibia tunnel 95, the anchor flips ninety degrees, as shown in FIG. 37 and first and second tag ends 60, 62 are pulled to tighten first and second adjustable loops 54a, 54b between suture button 100 and anchor 84. The self-cinching sections of suture construct 52 maintain the tension on the suture construct 52, and the first and second tag ends 60, 62 may be trimmed.

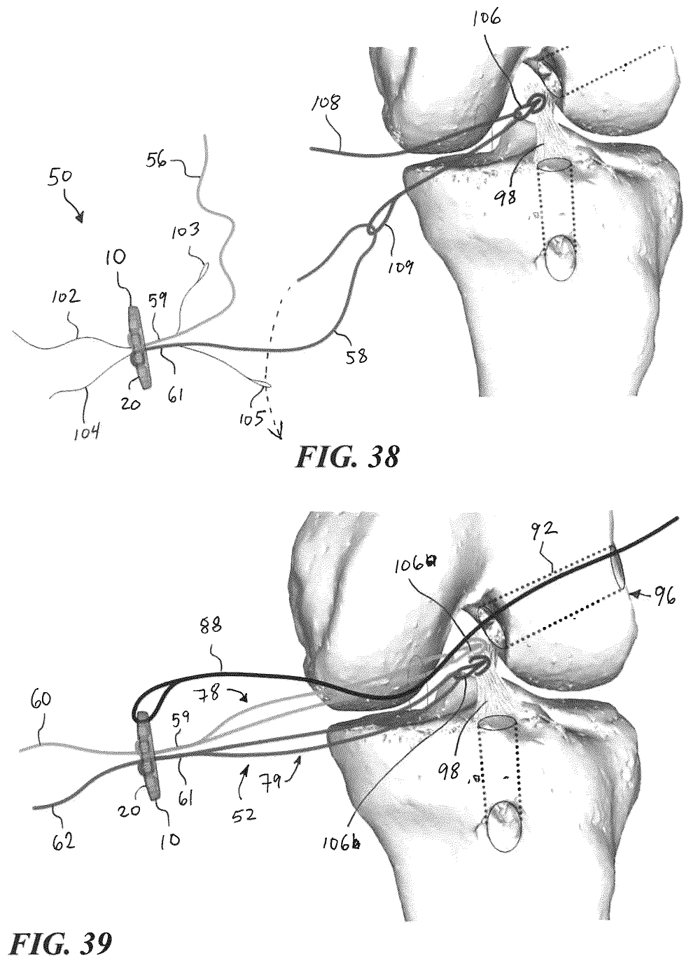

Referring to FIGS. 38-40, additional embodiments of a suture assembly 50 are illustrated for ACL repair. Suture assembly 50 includes a suture button 10 and a suture construct. As shown in FIG. 38, first strand 56 and second strand 58 are initially loose and have not yet been inserted through first and second self-cinching sleeves 59, 61, respectively. A first threading suture 102 is disposed through first self-cinching sleeve 59, and a second threading suture 104 is disposed through second self-cinching sleeve 61. A first threading loop 103 is disposed on first threading suture 102, and a second threading loop 105 is disposed on second threading suture 104. From this configuration, suture assembly 50 is configured for use in an ACL repair procedure. A torn or damaged ACL 98 for reattachment or repair is attached to suture assembly 50 using an attachment construct 106. Attachment construct 106 can include any suitable device for establishing an attachment point on the ACL 98. In some embodiments, attachment construct 106 includes a continuous loop traversing through the ACL tissue and forming a hitch with an open loop protruding from the ACL 90. An attachment suture 108 is passed through the open loop protruding from the attachment construct 106. Attachment suture 108 includes an open end loop 109. First or second strand 56, 58 is passed through the open end loop 109 in attachment suture 108. The free end of the first or second strand 56, 58 is passed through a corresponding threading loop 103, 105 in first or second strand 56, 58. From this position, shown in FIG. 38, second threading suture 104 is pulled axially through second self-cinching sleeve 61, thereby pulling the free end of second strand 58 in second threading loop 105 through second self-cinching sleeve 61 such that second tag end 62 extends from the top surface 20 of button 10. A similar procedure is performed with first threading suture 102 wherein the free end of first strand 56 is inserted into the first threading loop 103 in first threading suture 102, and first threading suture 102 is pulled through first self-cinching sleeve 59 in a direction away from top surface 20. As such, first and second tag ends 60, 62 extend away from top surface 20 on suture button 10. First and second attachment constructs 106a, 106b are attached to first and second adjustable loops 78, 79, shown in FIG. 39, and the assembly is ready for passage through the femoral tunnel 92.

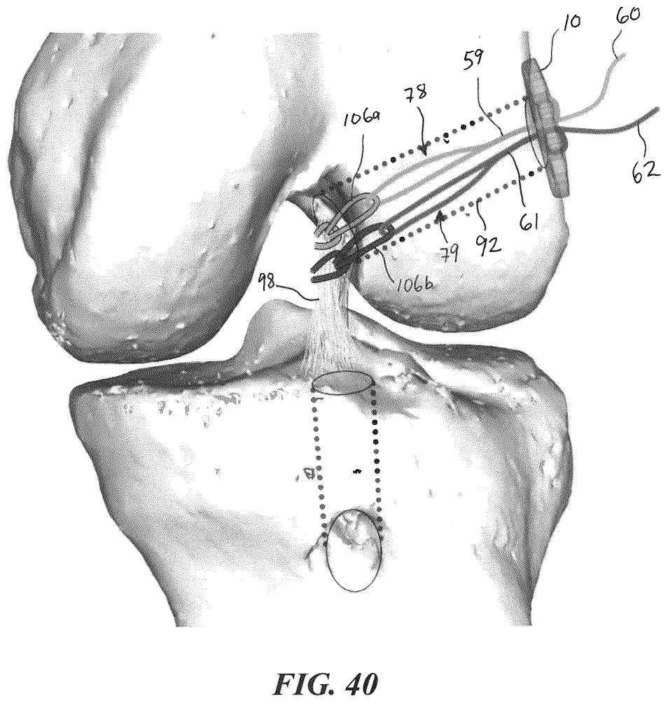

A passing suture 88 installed in first or second hole in suture button 10 is passed through femoral tunnel 92, shown in FIG. 39. From this position, the passing suture 88 is pulled in a direction away from the femoral cortex 96, thereby drawing the suture button 10 and attached suture construct 52 past the ACL 98 and into and through the femoral tunnel 92. Upon exiting the femoral tunnel 92 near the femoral cortex 96, the suture button 10 rotates to an angle parallel to the cortex, and the first and second tag ends 60, 62 may be pulled to tighten first and second adjustable loops 78, 79. As first and second tag ends 60, 62 are pulled, the first and second strands are able to slide through first and second attachment constructs 106a, 106b like a pulley and the ACL 96 is pulled toward and/or into the femoral tunnel into an anatomically correct position. The first and second tag ends 60, 62 are tightened further to apply the desired biomechanical tension to the ligament, and upon tightening, the tension is maintained via first and second self-cinching sleeves 59, 61. After tightening, first and second tag ends 60, 62 may be trimmed, and suture button 10 rests tightly against the femoral cortex, as shown in FIG. 40.

Thus, although there have been described particular embodiments of the present invention of a new and useful SELF-CINCHING ADJUSTABLE LOOP SUTURE BUTTON AND METHODS, it is not intended that such references to particular embodiments be construed as limitations upon the scope of this invention.

What is claimed is:

1. A suture button apparatus comprising:
a suture button including a top surface, a bottom surface, a first end and a second end;
a center passage defined through the suture button between the first and second ends;
a first passage defined through the suture button between the center passage and the first end;
a second passage defined through the suture button between the center passage and the second end;
a first bridge between the center passage and the first passage; and
a second bridge between the center passage and the second passage.

2. The apparatus of claim 1, further comprising:
a first ramp disposed on the top surface between the first passage and the first end.

3. The apparatus of claim 2, further comprising:
a second ramp disposed on the top surface between the first passage and the second end.

4. The apparatus of claim 3, further comprising:
a first hole defined in the suture button between the first passage and the first end.

5. The apparatus of claim 4, further comprising:
a second hole defined in the suture button between the second passage and the second end.

6. The apparatus of claim 5, further comprising:
a recess defined in the bottom surface, the recess open to the first passage, second passage and center passage.

7. The apparatus of claim 6, wherein the recess includes a rectangular profile into the suture button.

8. The apparatus of claim 7, wherein the center passage has a rectangular profile through the suture button.

9. The apparatus of claim 8, wherein the first passage has a rectangular profile through the suture button.

10. The apparatus of claim 9, wherein the second passage has a rectangular profile through the suture button.

11. The apparatus of claim 10, wherein the first bridge includes a rectangular cross-sectional profile.

12. The apparatus of claim 10, wherein the second bridge includes a rectangular cross-sectional profile.

13. The apparatus of claim 12, further comprising a self-cinching adjustable loop suture construct disposed on the suture button.

14. The apparatus of claim 13, wherein the self-cinching adjustable loop suture construct is disposed on the suture button by a hitch around first and second bridges.

15. The apparatus of claim 14, further comprising a cross strand of the self-cinching adjustable loop suture construct disposed in the recess.

16. The apparatus of claim 15, further comprising a passing suture disposed in the first hole.

17. A suture assembly apparatus, comprising;
a suture button including a first end, a second end, a top surface, a bottom surface, a first passage, a second passage, a center passage between the first and second passages, a first bridge between the center passage and the first passage and a second bridge between the center passage and the second passage;
a suture including a first strand positioned around the first bridge, a second strand positioned around the second bridge, and a cross strand extending between the first bridge and the second bridge;
a first self-cinching section on the suture;
a second self-cinching section suture;
a first adjustable loop formed in the first strand; and
a second adjustable loop formed in the second strand.

18. The apparatus of claim 17, further comprising:
the first self-cinching section disposed on the second strand extending into the center passage;
the second self-cinching section disposed on the first strand extending into the center passage;
wherein the first strand extends through the first self-cinching section in the second strand, and
wherein the second strand extends through the first self-cinching section in the first strand.

19. The apparatus of claim 17, further comprising:
the first self-cinching section disposed on the first strand, wherein the first strand extends through the first self-cinching section on the first strand; and
the second self-cinching section disposed on the second strand, wherein the second strand extends through the second self-cinching section on the second strand.
